(19) United States
(12) Patent Application Publication
(10) Pub. No.: US 2023/0255550 A1
(43) Pub. Date: Aug. 17, 2023

(54) DEVICES, SYSTEMS, AND METHODS FOR QUANTIFYING STABILITY OF A JOINT

(71) Applicant: Ohio State Innovation Foundation, Columbus, OH (US)

(72) Inventors: Anna LEE, South Lyon, MI (US); Robert SISTON, Dublin, OH (US); David C. FLANIGAN, Columbus, OH (US); Robert A. MAGNUSSEN, Worthington, OH (US); Robert DUERR, Columbus, OH (US)

(21) Appl. No.: 18/013,842

(22) PCT Filed: Jun. 30, 2021
(86) PCT No.: PCT/US21/39909

§ 371 Date: Dec. 29, 2022

Related U.S. Application Data
(60) Provisional application No. 63/046,429, filed on Jun. 30, 2020.

(51) Int. Cl. A61B 5/00; A61B 5/11

(52) U.S. Cl. CPC A61B 5/4585; A61B 5/6806; A61B 5/1127; A61B 2562/0252

(57) ABSTRACT

The present disclosure describes systems, devices, and methods for determining stability of a joint of a living subject. In one aspect, a force-measuring device for determining stability of a joint of a living subject is provided. In some examples, the force-measuring device includes a mitt frame configured to receive a hand of a user therein, one or more first force sensors, and one or more second force sensors. The mitt frame may include a palm portion configured to receive a palm of the user therein, and a finger portion configured to receive fingers of the user therein. The one or more first force sensors may be coupled to the palm portion and disposed on an exterior surface of the palm portion. The one or more second force sensors may be coupled to the finger portion and disposed on an exterior surface of the finger portion.

### CROSS-REFERENCE TO RELATED APPLICATION

This application claims the benefit of U.S. Provisional Application No. 63/046,429, filed Jun. 30, 2020, and titled DEVICE AND METHOD TO QUANTIFY KNEE STABILITY DURING ANTERIOR CRUCIATE LIGAMENT RECONSTRUCTION, the disclosure of which is expressly incorporated herein by reference in its entirety.

### FIELD

The present application generally is directed to devices, systems, and methods for medical assessment. More specifically, examples described herein relate to quantifying stability of a joint, such as a knee, of a living subject.

### BACKGROUND

In various scenarios, medical practitioners may seek to assess the stability of a joint of a patient. Joint stability assessment is often subjective, and stability determinations can vary from one practitioner to another. Stability assessment is often based on feel and determinations can vary based on experience and skill level of a medical practitioner performing the assessment.

One common example application where a medical practitioner will assess the stability of a joint is in assessment of the knee in relation to total knee arthroplasty (TKA). Arthritis and other rheumatic conditions are the leading cause of disability in the United States and are among the most common chronic disease problems in the country. A definitive treatment to relieve the pain, disability, and loss of motion associated with late-stage osteoarthritis is TKA. Over 750,000 primary TKAs were performed in 2014, and over 1.25 million primary TKAs are expected annually by 2030.

Unfortunately, not all patients of these surgeries experience consistently positive outcomes. While TKA successfully relieves the pain associated with osteoarthritis of the knee for most patients, more than 20% of adults demonstrate functional deficits 2-years post-TKA. Following TKA, patients have been observed to walk, rise from a chair, and climb stairs more slowly than age-matched controls and have stiff-knee gait. It is common for patients following TKA to be limited in the performance of common yet physically demanding activities such as stair negotiation, squatting, kneeling, gardening, and recreational sports.

Another common example application where a medical practitioner will assess the stability of a joint is in assessment of the knee in relation to the anterior cruciate ligament (ACL). Over 250,000 people tear their ACL in the United States each year. The anterior cruciate ligament is one of four primary ligaments in the knee and is responsible for restraining anterior displacement and internal rotation of the tibia. ACL injuries can happen in a wide range of people from recreational athletes to professional athletes, but ACL tears are most common in athletes in the range of ages 14 to 25 years. In addition, females are four to six times more likely to tear their ACL. Most ACL injuries occur during a non-contact event, where a player tears their ACL during motions such as cutting and pivoting. A valgus moment at the knee and internal rotation of the tibia are the most common motions that lead to an ACL tear. Knee valgus angles and moments are also primary predictors of ACL injury risk. This is common in sports like soccer, basketball, skiing, and football. A torn ACL results in altered kinematic and kinetic behavior of the leg. Increased variability in the kinematics of the injured knee and significantly different knee abduction angles occur after a person sustains an ACL injury. A change in kinematics and kinetic behavior can cause knee instability, which can lead to reduced sports participation after an ACL injury. Other negative outcomes of a torn ACL include quadriceps strength asymmetry between the injured and uninjured leg and development of early knee osteoarthritis. Several studies have looked at knee stability in patients who have undergone anterior cruciate ligament reconstruction (ACLR) compared to patients who have not had any previous knee injuries. One study showed that there was increased internal and external rotation during walking in patients who had undergone ACLR compared to people who have not torn their ACL. Another study also showed significant increased internal rotation of the uninjured knee of patients who have undergone contralateral ACLR compared to healthy individuals, Following ACL reconstruction, only 55% of patients return to a competitive level of sport, up to one-in-three did not return to any level of sport, and 79% reported their knee as the reason for not returning.

Placement of a graft on a patient's knee is an important decision made by a surgeon in relation to operations such as TKA and ACLR. The choice of placement can have lasting effects on the patient's health, and the ability to check for proper placement is important. After a graft is secured, the surgeon will check for a stable knee. There are several ways to clinically test for knee stability including the Lachman test, the pivot-shill test, and the anterior drawer test. These tests can also be used to identify when a patient has a torn ACL. The Lachman test is performed when the patient is lying supine with a flexed knee. The examiner places one hand on the shin and pulls anteriorly while holding the thigh with their other hand. A patient has a torn ACL and unstable knee when the tibia is pulled anteriorly, and the examiner feels no hard endpoint. The pivot-shift test assesses the rotational instability and anterior translation of the knee. The patient is lying supine with their legs in full extension and the examiner holds the heel or ankle of the patient's injured leg and holds the shin just below the knee with their other hand. The examiner then applies a valgus force to the knee and internally rotates the tibia while moving the knee into flexion. The examiner feels for how stable the knee is during these movements. In view of the subjective nature of these tests, there is a need to determine knee stability in a quantifiable way.

One way to quantify knee stability is by measuring three motions of the knee due to an applied load: varus/valgus rotation, internal/external rotation, and anterior/posterior translation. A moment or force is applied in each direction and the resulting displacement is measured. These two values are plotted to create a stability curve and find the knee laxity and terminal stiffness. Knee laxity is the region of the stability curve where large displacements and rotations occur under low applied forces and moments. Terminal stiffness is the slope of the end region of the stability curve where large, applied forces and moments result in small displacements and rotations. Certain devices have been used to determine knee laxity, some of which may require a patient to lay in a prone position that is not conducive to certain operations such as ACLR. Some devices are usable during diagnostic procedures but are not serializable for use during surgical operation or are undesirably large for use in an operating room, Previous devices for determination of knee laxity rigidly fix a knee in place and mount the patient's limbs to a measurement device. An example device has been disclosed in U.S. Pat. No. 8,888,715, which is incorporated by reference herein in its entirety.

There remains a need for a sterilizable, portable device that can be used to quantify knee laxity and stiffness in multiple directions during diagnostic procedures as well as medical operations.

### SUMMARY

The present disclosure describes systems, devices, and methods for determining stability of a joint of a living subject. In one aspect, a force-measuring device for determining stability of a joint of a living subject is provided. In some examples, the force-measuring device includes a mitt frame configured to receive a hand of a user therein, one or more first force sensors, and one or more second force sensors. The mitt frame may include a palm portion configured to receive a palm of the user therein, and a finger portion configured to receive fingers of the user therein. The one or more first force sensors may be coupled to the palm portion and disposed on an exterior surface of the palm portion. The one or more second force sensors may be coupled to the finger portion and disposed on an exterior surface of the finger portion.

In some examples, the mitt frame is formed of one or more sterilizable materials.

In some examples, the finger portion is movably coupled to the palm portion.

In some examples, the finger portion is rotatably coupled to the palm portion.

In some examples, the force-measuring device also includes a rotary encoder coupled to the finger portion and the palm portion and configured to track a location of the finger portion relative to the palm portion.

In some examples, the finger portion is removably coupled to the palm portion.

In some examples, the palm portion includes a palmar component and a dorsal component coupled to one another.

In some examples, the palmar component of the palm portion is formed of a first material, the dorsal component of the palm portion is formed of a second material, and the first material is more flexible than the second material.

In some examples, the palmar component of the palm portion includes one or more pockets, and the one or more first force sensors are disposed at least partially within the one or more pockets.

In some examples, the palm portion includes a plurality of interconnected struts defining a plurality of openings therebetween, and each of the openings extends from the exterior surface to an interior surface of the palm portion.

In some examples, the finger portion includes a palmar component and a dorsal component coupled to one another.

In some examples, the palmar component of the finger portion is formed of a first material, the dorsal component of the finger portion is formed of a second material, and the first material is more flexible than the second material.

In some examples, the palmar component of the finger portion includes one or more pockets, and the one or more second force sensors are disposed at least partially within the one or more pockets.

In some examples, the finger portion includes a plurality of interconnected struts defining a plurality of openings therebetween, and each of the openings extends from the exterior surface to an interior surface of the finger portion.

In some examples, the one or more first force sensors are disposed on a palmar side of the palm portion.

In some examples, the one or more first force sensors are removably coupled to the palm portion.

In some examples, the one or more first force sensors include one or more load cells.

In some examples, the one or more first force sensors include one or more piezoelectric sensors.

In some examples, the one or more first force sensors include one or more piezoresistive sensors.

In some examples, the one or more first force sensors include one or more force plates.

In some examples, the one or more force plates include one or more load cells disposed between a pair of plates.

In some examples, the one or more second force sensors are disposed on a palmar side of the finger portion.

In some examples, the one or more second force sensors are removably coupled to the finger portion.

In some examples, the one or more second force sensors include one or more load cells.

In some examples, the one or more second force sensors include one or more piezoelectric sensors.

In some examples, the one or more second force sensors include one or more piezoresistive sensors.

In some examples, the one or more second force sensors include one or more force plates.

In some examples, the one or more force plates include one or more load cells disposed between a pair of plates.

In some examples, the force-measuring, device also includes a strap coupled to the palm portion and configured to removably secure the mitt frame to a wrist of the user.

In some examples, the force-measuring device also includes an electronics module in operable communication with the one or more first force sensors and the one or more second force sensors. The electronics module may be configured to receive force data from the one or more first force sensors and the one or more second force sensors.

In some examples, the electronics module is in operable communication with the one or more first force sensors and the one or more second force sensors via wires disposed along a dorsal side of the mitt frame.

In some examples, the electronics module includes one or more breadboards, one or more multiplexers, and one or more computing device.

In some examples, the force-measuring device also includes a strap coupled to the electronics module and configured to removably secure the electronics module to a forearm of the user.

In some examples, the force-measuring device also includes a plurality of mitt tracking markers coupled to the mitt frame and disposed on an exterior surface of the mitt frame.

In some examples, the mitt tracking markers are coupled to the finger portion.

In some examples, the mitt tracking markers are coupled to the palm portion.

In some examples, the mitt tracking markers include passive optical tracking markers.

In some examples, the mitt frame also includes a thumb portion configured to receive a thumb of the user therein.

In some examples, the thumb portion is movably coupled to the palm portion.

In some examples, the thumb portion is rotatably coupled to the palm portion.

In some examples, the force-measuring device also includes a rotary encoder coupled to the thumb portion and the palm portion and configured to track a location of the thumb portion relative to the palm portion.

In some examples, the thumb portion is removably coupled to the palm portion.

In some examples, the force-measuring device also includes one or more third force sensors coupled to the thumb portion and disposed on an exterior surface of the thumb portion.

In some examples, the one or more third force sensors are disposed on a palmar side of the thumb portion.

In some examples, the one or more third force sensors are removably coupled to the thumb portion.

In some examples, the one or more third force sensors include one or more load cells.

In some examples, the one or more third force sensors include one or more piezoelectric sensors.

In some examples, the one or more third force sensors include one or more piezoresistive sensors.

In some examples, the one or more third force sensors include one or more force plates.

In some examples, the one or more force plates include one or more load cells disposed between a pair of plates.

In another aspect, a system for determining stability of a joint of a living subject is provided. In some examples, the system includes a force-measuring device and a displacement-tracking system. The force-measuring device may include a mitt frame configured to receive a hand of a user therein, one or more first force sensors, and one or more second force sensors. The mitt frame may include a palm portion configured to receive a palm of the user therein, and a finger portion configured to receive fingers of the user therein. The one or more first force sensors may be coupled to the palm portion and disposed on an exterior surface of the palm portion. The one or more second force sensors may be coupled to the finger portion and disposed on an exterior surface of the finger portion. The displacement-tracking system may be configured to track position and orientation of the force-measuring device and position and orientation of one or more body parts associated with the joint of the subj ea.

In some examples, the force-measuring, device also includes a plurality of mitt tracking markers coupled to the mitt frame and disposed on an exterior surface of the mitt frame, and the displacement-tracking, system is configured to track position and orientation of the force-measuring device by tracking position and orientation of the mitt tracking markers.

In some examples, the mitt tracking markers include passive optical tracking markers.

In some examples, the displacement-tracking system includes a plurality of subject tracking markers configured to be removably coupled to the one or more body parts associated with the joint of the subject.

In some examples, the subject tracking markers include passive optical tracking markers.

In some examples, the subject tracking markers include a first subject tracking marker configured to be removably coupled to a first body part associated with the joint of the subject and a second subject tracking marker configured to be removably coupled to a second body part associated with the joint of the subject.

In some examples, the displacement-tracking system is configured to track position and orientation of the one or more body parts associated with the joint of the subject by tracking position and orientation of the subject tracking markers.

In some examples, the displacement-tracking system includes one or more cameras, one or more signal processing units, and one or more computing devices.

In some examples, the displacement-tracking system includes a surgical navigation system.

In some examples, the system also includes a computing device in operable communication with the force-measuring device and the displacement-tracking system.

In some examples, the computing device is configured to receive subject position and orientation data from the displacement-tracking system, the subject position and orientation data being indicative of positions and orientations of the one or more body parts associated with the joint of the subject. The computing device also may be configured to determine, based at least in part on the subject position and orientation data, subject displacement data indicative of rotational and translational displacements of the one or more body parts associated with the joint of the subject.

In some examples, the computing device is further configured to receive force data from the force-measuring device, the force data being indicative of forces applied by the user to the joint of the subject via the force-measuring device. The computing device also may be configured to receive mitt position and orientation data from the displacement-tracking system, the mitt position and orientation data being indicative of positions and orientations of the mitt frame. The computing device also may be configured to determine, based at least in part on the force data and the mitt position and orientation data, moment data indicative of moments about the joint of the subject resulting from the forces applied by the user.

In some examples, the computing device is further configured to determine, based at least in part on the subject displacement data, the force data, and the moment data, one or more stability values indicative of stability of the joint of the subject.

In another aspect, a method for determining stability of a joint of a living subject is provided. The method may include determining, via a force-measuring device, force data indicative of forces applied by a user to the joint of the subject via the force-measuring device. The force-measuring device may include a mitt frame configured to receive a hand of the user therein, and one or more force sensors coupled to the mitt frame. The method also may include determining, via a displacement-tracking system, subject position and orientation data indicative of positions and orientations of one or more body parts associated with the joint of the subject. The method also may include determining, based at least in part on the subject position and orientation data and the force data, one or more stability values indicative of stability of the joint of the subject.

In some examples, the mitt frame includes a palm portion configured to receive a palm of the user therein, and a finger portion configured to receive fingers of the user therein.

In some examples, the one or more force sensors includes one or more first force sensors coupled to the palm portion and disposed on an exterior surface of the palm portion, and one or more second force sensors coupled to the finger portion and disposed on an exterior surface of the finger portion.

In some examples, the mitt frame also includes a thumb portion configured to receive a thumb of the user therein.

In some examples, the one or more force sensors also includes one or more third force sensors coupled to the thumb portion and disposed on an exterior surface of the thumb portion.

In some examples, the displacement-tracking system includes one or more cameras, one or more signal processing units, and one or more computing devices.

In some examples, the displacement-tracking system includes a surgical navigation system.

In some examples, determining the force data includes receiving force signals from the one or more force sensors.

In some examples, determining the subject position and orientation data includes tracking positions and orientations of a plurality of subject tracking markers removably coupled to the one or more body parts associated with the joint of the subject.

In some examples, the method also includes determining, based at least in part on the subject position and orientation data, subject displacement data indicative of rotational and translational displacements of the one or more body parts associated with the joint of the subject.

In some examples, determining the one or more stability values includes determining the one or more stability values based at least in part on the subject displacement data and the force data.

In some examples, the method also includes determining, via the displacement-tracking system, mitt position and orientation data indicative of positions and orientations of the mitt frame.

In some examples, determining the mitt position and orientation data includes tracking positions and orientations of a plurality of mitt tracking markers coupled to the mitt frame.

In some examples, determining the one or more stability values includes determining the one or more stability values based at least in part on the subject position and orientation data, the force data, and the mitt position and orientation data.

In some examples, the method also includes determining, based at least in part on the force data and the mitt position and orientation data, moment data indicative of moments about the joint of the subject resulting from the forces applied by the user.

In some examples, determining the one or more stability values includes determining the one or more stability values based at least in part on the subject position and orientation data, the force data, and the moment data.

In some examples, the joint is a knee of the subject.

In some examples, the method is performed during an anterior cruciate ligament reconstruction procedure, a total knee arthroplasty procedure, a medial patellofemoral ligament reconstruction procedure, a posterior cruciate ligament reconstruction procedure, a medial collateral ligament reconstruction procedure, or a lateral collateral ligament reconstruction procedure.

In some examples, the method is performed within an operating room.

### BRIEF DESCRIPTION OF THE DRAWINGS

FIG. 1 is a schematic diagram of an example system for determining stability of a joint of a living subject.

FIG. 2 is a perspective view of an example force-measuring device as may be used for the system of FIG. 1.

FIG. 3 is a perspective view of the force-measuring device of FIG. 2.

FIG. 4 is a palmar view of the force-measuring device of FIG. 2.

FIG. 5 is a dorsal view of the force-measuring device of FIG. 2.

FIG. 6 is a perspective view of an example force sensor as may be used for the force-measuring device of FIG. 2, showing four load cells.

FIG. 7 is a top view schematic diagram and a side view schematic diagram of the force sensor of FIG. 6.

FIG. 8 is a schematic diagram of example electronic components as may be used for the force-measuring device of FIG. 2.

FIG. 9 is a schematic diagram of example electronic components as may be used for the force-measuring device of FIG. 2.

FIG. 10 is a perspective view of an example passive optical tracking marker.

FIG. 11 is a perspective view of a pair of example passive tracking markers coupled to a tibia and a femur.

FIG. 12 is a flow diagram of an example process for a knee stability algorithm.

FIG. 13 is a schematic diagram of an example: computing device.

### DETAILED DESCRIPTION

In the following description, specific details are set forth describing some examples consistent with the present disclosure. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, and components have not been described in detail so as not to unnecessarily obscure aspects of the examples.

Examples of force-measuring devices, systems, and methods for determining stability of a joint of a living subject are described herein. A force-measuring device generally may include a mitt frame, one or more first force sensors, and one or more second force sensors. The mitt frame may be configured to receive a hand of a user therein and may include different portions for receiving respective portions of the user's hand. For example, the mitt frame may include a palm portion for receiving the user's palm and a finger portion for receiving the user's fingers. The first force sensor(s) may be coupled to the palm portion and disposed on an exterior surface of the palm portion, while the second force sensor(s) may be coupled to the finger portion and disposed on an exterior surface of the finger portion. In some examples, the mitt frame also may include a thumb portion for receiving the user's thumb, and the force-measuring device also may include one or more third force sensors coupled to the thumb portion and disposed on an exterior surface of the thumb portion.

The force-measuring device may be used to measure forces and the location of forces applied to a patient's joint to facilitate assessment of joint stability. Although the force-measuring device may be particularly well-suited for assessing stability of a patient's knee, it will be appreciated that the force-measuring device may be used for stability assessment of other types of anatomical joints in various applications. In contrast to existing devices that are effectively limited to use in a diagnostic setting, the force-measuring device may be used to assess joint stability in a surgical setting. For example, the force-measuring device may be used in an operating room while not significantly impeding how physicians perform operations such as ACLR, TKA, medial patellofemoral ligament reconstruction (MPLR), posterior cruciate ligament reconstruction (PCLR), Medial collateral ligament reconstruction (MCLR), or lateral collateral ligament reconstruction (LCLR).

In the context of assessing a patient's knee, the force-measuring devices, systems, and methods provided herein advantageously allow medical practitioners to be able to replicate their normal knee stability testing procedures, such as the Lachman test and pivot-shift test, while utilizing a device that can quantify knee laxity. The configuration of the mitt frame and the force sensors provides a tool that requires users to make minimal adjustments to operations such as ACLR, TKA, MPLR, MCLR, and LCLR while allowing the users to quantity knee stability, Additionally, the configuration of the force-measurement devices and overall systems allows a user to apply forces wherever they prefer on a patient's leg and to maintain the “feel” of traditional knee stability tests like the Lachman test and the pivot-shift exam. The force-measurement device can be easily put on by a user to assess knee stability, and the device can be removed before continuing with additional steps of a procedure such as a surgery. This allows a practitioner to quantify knee stability in any direction of interest during an operation. It will be appreciated that the force-measuring devices, systems, and methods described herein may be used to assess stability of other types of anatomical joints to provide similar benefits.

As discussed herein, the force-measuring device may be used with a displacement-tracking system that is configured to track position and orientation of the force-measuring device. The displacement-tracking system may include one or more cameras, one or more signal processing units, and one or more computing devices. The camera(s) may be used to track position and orientation of the force-measuring device. For example, the force-measuring device may include mitt tracking markers coupled to the mitt frame, which allow the displacement-tracking system to track position and orientation of the mitt frame. The displacement-tracking system also may be configured to track position and orientation of one or more body parts associated with the subject's joint. For example, the displacement-tracking system may include subject tracking markers configured to be removably coupled to the body part(s), which allow the displacement-tracking system to track position and orientation of the body part(s). During assessment of stability of the subject's joint, the force-measuring device may be used to obtain force data indicative of forces applied by the user to the joint via the force-measuring device. Meanwhile, the displacement-tracking system may be used to obtain mitt position and orientation data indicative of positions and orientations of the mitt frame and also to obtain subject position and orientation data indicative of positions and orientations of the body part(s). The subject position and orientation data may be used to determine subject displacement data indicative of rotational and translational displacements of the body part(s). The force data and the mitt position and orientation data may be used to determine moment data indicative of moments about the subject's joint resulting from forces applied by the user. Ultimately, the subject displacement data may be used with the force data and/or the moment data to determine one or more stability values indicative of stability of the subject's joint. In this manner, the force-measuring devices, systems, and methods provided herein may allow the user to quantify stability of the subject's joint.

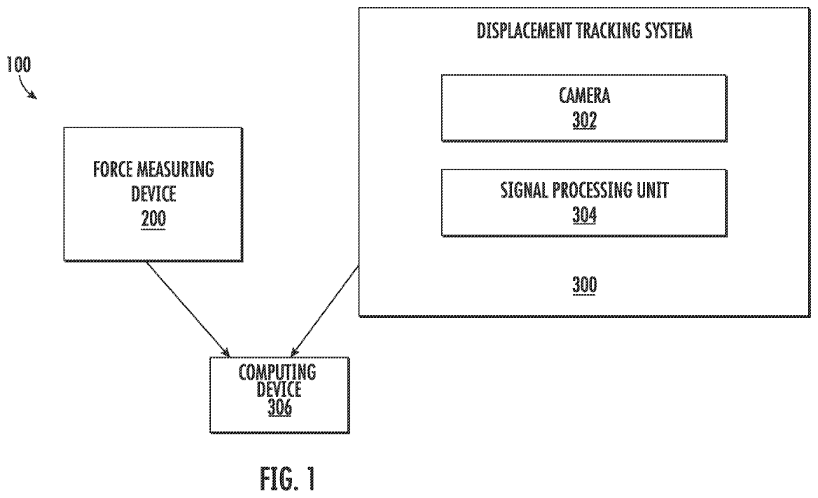

FIG. 1 shows an example system 100 for determining stability of a joint of a living subject. In some examples, the system 100 includes a force-measuring device 200 and a displacement-tracking system 300.

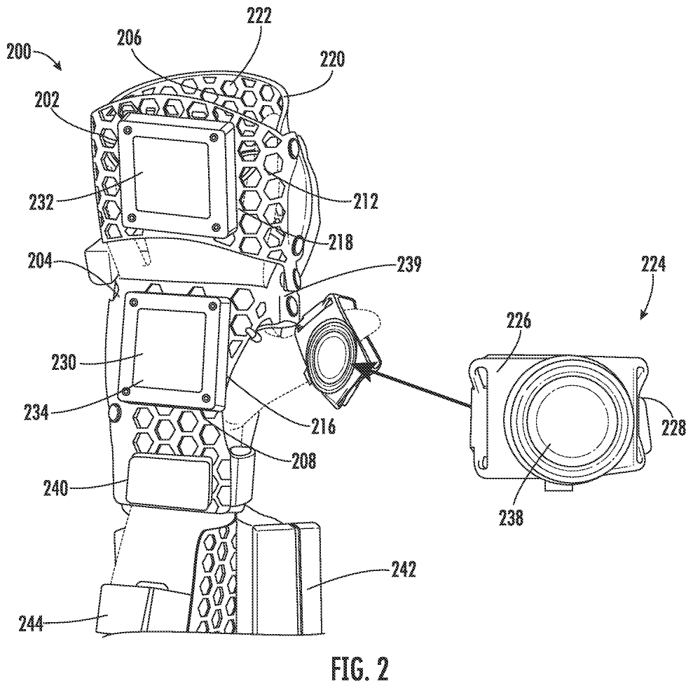

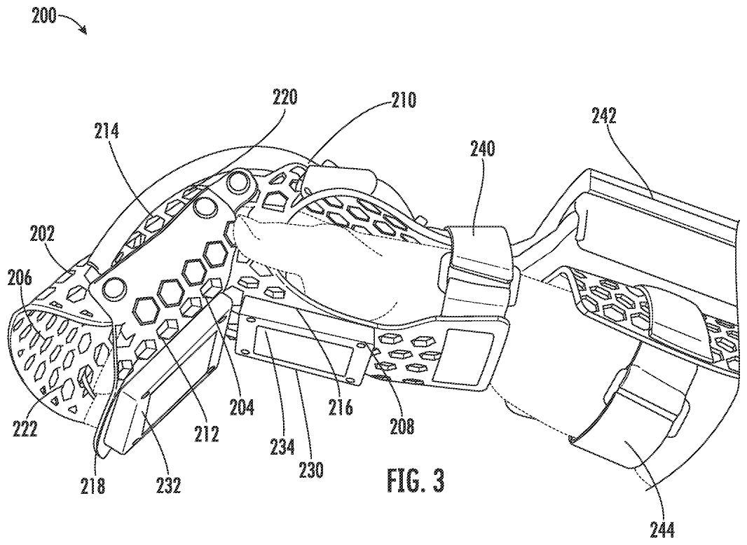

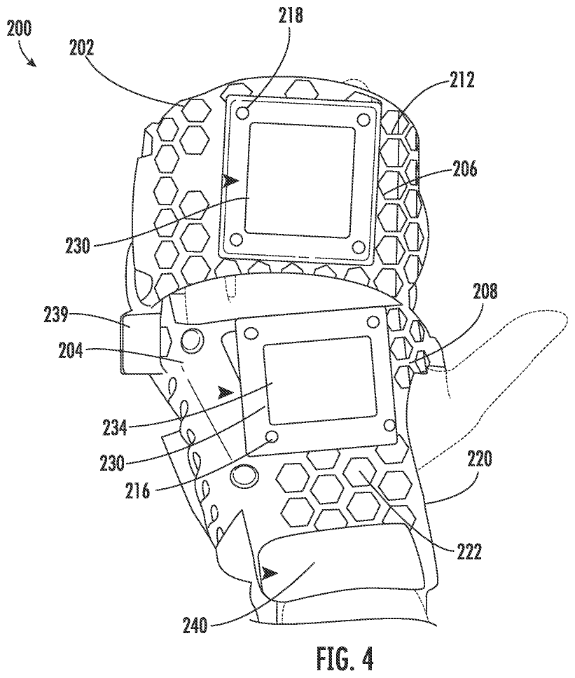

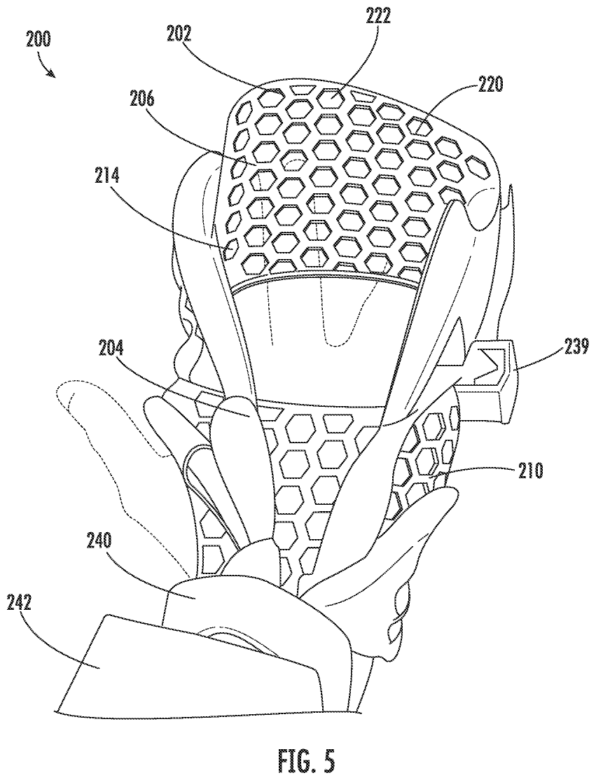

FIGS. 2-5 show examples of the force-measuring device 200. In some examples, the force-measuring device 200 includes a mitt frame 202, a first force sensor 230, a second force sensor 232, and a third force sensor coupled to the mitt frame 202. In some examples, the force-measuring device 200 also includes a rotary encoder 239 coupled to the mitt frame 202, a strap 240 coupled to the mitt frame 202, and an electronics module 242.

The mitt frame 202 provides a portable sensor platform that can be worn on a physician's hand during a surgery, examination, or other medical procedure. The mitt frame 202 can be put on and taken off during a surgical operation or other medical procedure without requiring adjustment or transportation of a patient. As described further below, the mitt frame 202 uses rigid and flexible parts that allow for motion tracking of the force-measuring device 200. Force sensors and motion trackers can be coupled to and removed from the mitt frame 202 such that the mitt frame 202 can be sterilized separately from the sensors and trackers.

In some examples, the mitt frame 202 includes a palm portion 204 and a finger portion 206. The palm portion 204 can receive a user's palm therein to move with and track the motion of the user's palm. The palm portion 204 is further provided to allow a user to manipulate a patient with the palm portion 204 and take measurements of the forces applied using the palm. In some examples, the palm portion 204 includes a palmar component 208 and a dorsal component 210 coupled to the palmar component 208 opposite and spaced apart from the palmar component 208 defining a space such that a user's hand can fit therebetween. The palmar component 208 and the dorsal component 210 of the palm portion 204 are movable with respect to each other such that the space between the palmar component 208 and the dorsal component 210 can be adjusted to accommodate various hand sizes. But in some examples, the space between the palmar component 208 and the dorsal component 210 is fixed. The palmar component 208 and the dorsal component 210 are connected to each other with barrel nuts and bolts, which promote smooth outer surfaces. In some examples, the palm portion 204 includes a pocket 216 that protrudes from the palmar component 208 and away from the dorsal component 210, such that at least one force sensor can be disposed therein. For example, force plates (described in more detail below) can be secured to the pocket 216 by at least one screw such that the force plates do not protrude beyond the pocket 216 providing a smooth surface such that no rough edges of the force plate contact a user's hand or the patient's leg. In some examples, the palm portion 204 includes a plurality of pockets. The palm portion 204, as shown in the example of FIGS. 2-5, includes a plurality of interconnected struts 220 defining a plurality of openings 222 therebetween. Each of the openings 222 extends from an exterior surface to an interior surface of the palm portion 204 such that objects and fluids can freely pass through each of the openings 222. But, in other examples, the palm portion 204 is a continuous surface, or any other surface suitable to be worn on a user's palm during a medical procedure.

The finger portion 206 is provided to receive a user's fingers therein to move with and track the motion of the user's fingers. The finger portion 206 is further provided to allow a user to manipulate a patient with the finger portion 206 and take measurements of the forces applied using the fingers. In some examples, the finger portion 206 includes a palmar component 212 and a dorsal component 214 coupled to the palmar component 212 opposite and spaced apart from the palmar component 212 defining a space such that a user's fingers can fit therebetween. In some examples, the palmar component 212 and the dorsal component 214 of the finger portion 206 are movable with respect to each other such that the space between the palmar component 212 and the dorsal component 214 can be adjusted to accommodate various hand sizes. But in some other examples the space between the palmar component and the dorsal component 214 is fixed. In some examples, the palmar component 212 and the dorsal component 214 are connected to each other with barrel nuts and bolts, which further promote smooth outer surfaces. In some examples, the pocket 218 protrudes from the palmar component 212 and away from the dorsal component 214, such that at least one force sensor can be disposed therein. For example, force plates (described in more detail below) can be secured to the pocket 218 by at least one screw such that the force plates do not protrude beyond the pocket 218 providing a smooth surface such that no rough edges of the force plate contact a user's hand or the patient's leg. In some examples, the finger portion includes a plurality of pockets. The finger portion 206 as shown in the example of FIGS. 2-5, includes a plurality of interconnected struts 220 defining a plurality of openings 222 therebetween. Each of the openings 222 extends from an exterior surface to an interior surface of the finger portion 206 such that objects and fluids can freely pass through each of the openings 222. But, in other examples, the finger portion 206 is a continuous surface, or any other surface suitable to be worn on a user's fingers during a medical procedure.

In some examples, the finger portion 206 is removably coupled to the palm portion 204, such that the finger portion 206 can be separated and reattached to the palm portion 204 for maintenance, sterilization, or versatility of application. In some examples, the finger portion 206 is also rotatably coupled to the palm portion 204 such that the finger portion 206 can be moved toward and away from the palm portion 204 as a user retracts or extends their hand. In the example shown in FIGS. 2-5 the palm portion 204 and the finger portion 206 are coupled with a barrel nut and bolt on the medial side to form a revolute joint, which allows a user to move their fingers and palm. But in other examples, the finger portion 206 is otherwise moveably coupled to the palm portion 204 (e.g., slidably moveable) such that the finger portion 206 and the palm portion 204 are moveable toward and away from each other. In some examples, the palm portion 204 and the finger portion 206 are coupled by a hinge, fabric, or any other connector suitable to allow bending of a hand that is in the palm portion 204 and the finger portion 206.

In the example shown in FIGS. 2-5 the palmar components 208, 212 of the palm portion 204 and the finger portion 206 are each formed from Thermoplastic Polyurethane while the dorsal component 210, 214 of the palm portion 204 and the finger portions 206 are formed from Polylactic Acid. As such, the palmar components 208, 212 of the palm portion 204 and the finger portion 206 are formed from a material that is more flexible than the dorsal components 212, 214 of the palm portion 204 and the finger portion 206. But, in other examples the palmar components 208, 212 and the dorsal components 212, 214 of the palm portion 204 and the finger portion 206 are formed from any other sterilizable material suitable to be worn by a user during a procedure or examination.

FIG. 2 shows an example of the thumb portion 224. The thumb portion 224 is provided to receive a thumb of the user therein and track the motion of the user's thumb. The thumb portion 224 is further provided to allow a user to manipulate a patient with the thumb portion 224 and take measurements of the forces applied using the thumb. In some examples, the thumb portion 224 includes a palmar component 226 and a dorsal component 228 coupled to the palmar component 226 opposite and spaced apart from the palmar component 226 and defining a space therebetween. In some examples, the palmar component 226 and the dorsal component 228 of the thumb portion 224 are movable with respect to each other such that the space between the palmar component 226 and the dorsal component 228 can be altered to accommodate various hand sizes. Although, in some other examples the space between the palmar component 226 and the dorsal component 228 is fixed. In the example shown in FIG. 2, the palmar component 226 and the dorsal component 228 are each formed from Polylactic Acid.

In some examples, the thumb portion 224 is removably coupled to the palm portion 204, such that the thumb portion 224 can be separated and reattached to the palm portion 204 for maintenance, sterilization, or versatility of application. In some examples, such as the example shown in FIG. 2 the thumb portion 224 is also rotatable and coupled to the palm portion 204 such that the thumb portion 224 can be moved toward and away from the palm portion 204 as a user compresses or extends their hand. But, in other examples, the thumb portion 224 is otherwise moveably coupled to the finger portion 206 (e.g., slidably moveable) such that the thumb portion 224 and the hand portion are moveable toward and away from each other. In some examples, such as the example shown in FIG. 2 the thumb portion 224 is coupled to the finger portion 206 by fabric, but, in other examples, the palm portion 204 and the finger portion 206 are coupled by a hinge, a bolt, or any other form of connector suitable to allow bending of a hand that is in the palm portion 204, the finger portion 206, and the thumb portion 224.

Although in the example shown in FIG. 2 the palmar component 226 of the thumb portion 224 and the dorsal component 228 of the thumb portion 224 are each formed from Polylactic Acid, in some examples the palmar component and the dorsal component of the thumb portion 224 are formed from any other sterilizable material suitable to be worn by a user (luting a procedure or examination. Although in the example shown in FIGS. 2-5 the palmer component 226 and the dorsal component 228 of the thumb portion 224 are each formed from the same material, in other examples the palmar component and the dorsal component 228 of the thumb portion 224 are each formed from different materials.

In some examples, the rotary encoder 239 is provided to track the relative angular position between the palm portion 204 and the finger portion 206, and a second rotary encoder 239 is provided to track the relative angular position between the thumb portion 224 and the palm portion 204. In some examples, each rotary encoder 239 converts the angular position of the palm portion 204 and the finger portion 206 relative to each other to an electrical signal such that the angular position of the objects can be transmitted to a computing device. In some examples, each rotary encoder 239 includes two portions that rotate relative to each other. One of the two portions is coupled a lateral side of the palm portion 204 and the other of the two portions of the rotary encoder 239 is coupled to a lateral side of the finger portion 206 or the thumb portion 224 to track the location of the palm portion 204 relative to the finger portion 206 or the thumb portion 224.

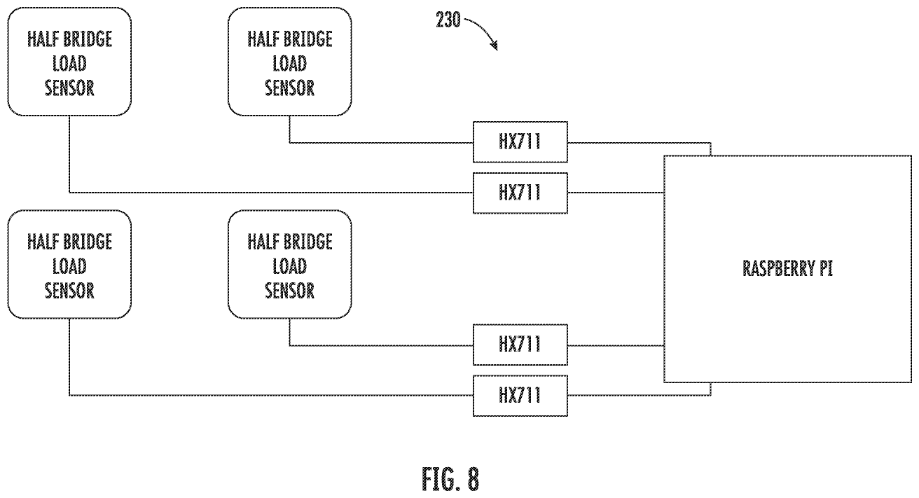

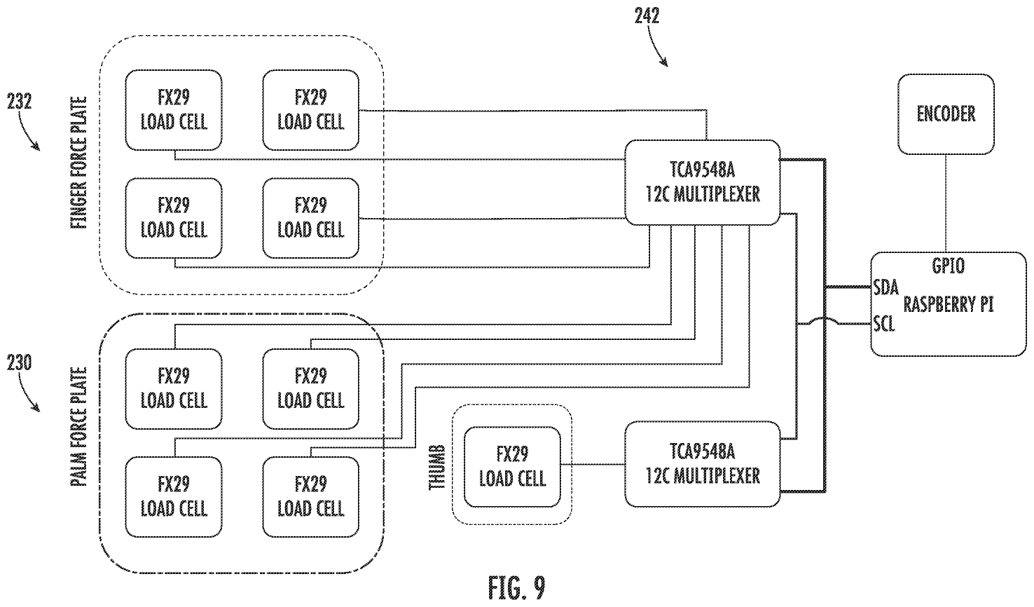

FIGS. 2-5 show examples that include the first force sensor 230 and the second force sensor 232 provided to measure force applied against the palm portion 204 or the finger portion 206. For example, the first force sensor 230 can be pressed against a first portion of a leg, to measure resistive forces in a first direction. The second force sensor 232 can be pressed against a second portion of a leg opposite the first portion of the leg, to measure resistive forces in a second direction. In some examples, the first force sensor 230 and the second force sensor 232 are each a portable force plate. In some examples, each force plate includes two panels 234 that are opposite and spaced apart from each other and four load cells 236 disposed between the panels 234. In some examples, the load cells 236 are TE Connectivity FX29 load cells which have a range of 100 lbf and a resolution of 0.006 lbf. But, in other examples, the load cells 236 can be any pressure sensor capable of measuring hand applied pressure. The load cells 236 may measure both static and dynamic measurements. In some examples, each of the load sensors include half bridge load sensors that can measure up to about 50 kg, although in other examples, load sensors having any suitable capacity can are used. Each of the load cells 236 are operably coupled together. For example, in the example shown in FIGS. 8-9 each cell is connected to two 1kΩ resistors to form a Wheatstone bridge. The load cells 236 are further connected to AVIA Semiconductor HX711 chips. The HX711 is a multiplexer, a programmable gain amplifier, a power supply regulator, and a 24-bit analog to digital converter. Each HX711 chip is electrically coupled to GPIO pins on a Raspberry Pi.

In some examples, the force plate of the first force sensor 230 is removably coupled to the palmar side of the palm portion 204 and disposed within the pocket 216 on the palm portion 204. In some examples, the force plate of the second force sensor 232 is removably coupled to the palmar side of the finger portion 206 and disposed within the pocket 218 on the finger portion 206. In some examples, each force plate uses four low profile load cells 236.

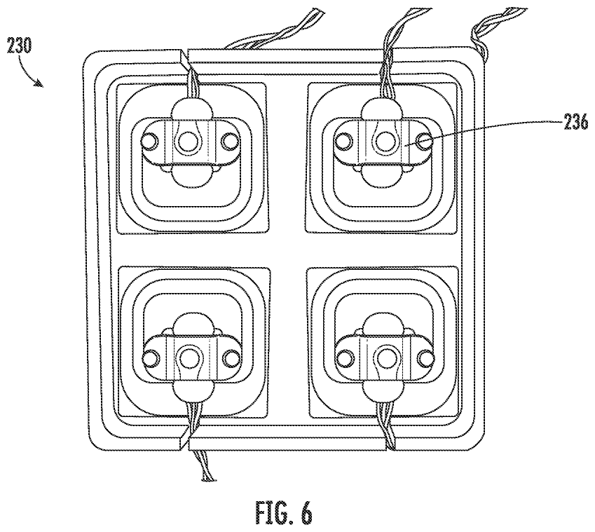

Although the force plate of the first force sensor 230 and the second force sensor 232 as shown in FIGS. 1-9 include four load cells 236, in some examples, each force plate can include any number of load cells disposed between the plates that is suitable for determining the laxity of a joint. Although in the example shown in FIGS. 2-5, the first force sensor 230 and the second force sensor 232 are each a force plate, in other examples, the first force sensor 230 and the second force sensor 232 each a different type of force sensor distinct from each other. Although the first force sensor 230 and the second force sensor 232 are each a force plate, in some examples each first force sensor includes one or more load cells, one or more piezoelectric sensors, one or more piezoresistive sensors. Although the example shown in FIGS. 2-5 the force-measuring includes one first force sensor 230 and one second force sensor 232, in some examples the force-measuring device 200 includes a plurality of first force sensors 230 and/or second force sensors 230, 232. Although the force plate of the first force sensor 230 is removably coupled to the palm portion 204 and disposed within the pocket 216 of the palm portion 204, in some examples the first force sensor 230 is fixedly coupled to the palm portion 204, or otherwise coupled to the palm portion 204. For example, in some examples the first force sensor 230 is coupled using reflowing or fasteners. Although the force plate of the second force sensor 232 is removably coupled to the finger portion 206 and disposed within the pocket 218 of the finger portion 206, in some examples the second force sensor 232 is fixedly coupled to the finger portion 206, or otherwise coupled to the finger portion 206. In some examples, the second force sensor 232 is coupled to the finger portion 206 using reflowing, fasteners, or any other coupling method that is suitable to attach a force sensor to a portable glove.

In some examples, such as the example shown in FIG. 2 the third force sensor 238 is a load cell that is removably coupled to the palmar side of the thumb portion 224 and is disposed on the exterior of the thumb portion 224.

Although in the example shown in FIG. 2 the third force sensor 238 is a load cell, in some examples the third force sensor 238 includes a plurality of load cells or other force sensors. For example, in some examples each third force sensor 238 includes one or more force plates such as force plates including one or more load cells disposed between a pair of plates, one or more piezoelectric sensors, or one or more piezoresistive sensors.

Although the force plate of the first force sensor 230 is removably coupled to the thumb portion 224, in some examples the third force sensor 238 is fixedly coupled to the thumb portion 224, or otherwise coupled to the thumb portion 224. For example, in some examples the third force sensor is coupled using reflowing or fasteners.

FIGS. 2-5 show the strap 240. The strap 240 removably secures the force-measuring device 200 to the wrist of a user. The strap 240 is a hook and loop fastener that can be disposed about a user when the user is using the mitt. The strap 240 is coupled to the palm portion 204 of the mitt frame 202 away from the finger portion 206. Although the strap 240 is a hook and loop fastener, in other examples, the strap 240 can include a clip, a snap, or any other fastener suitable to fasten a mitt like device about a user's arm, wrist, or hand.

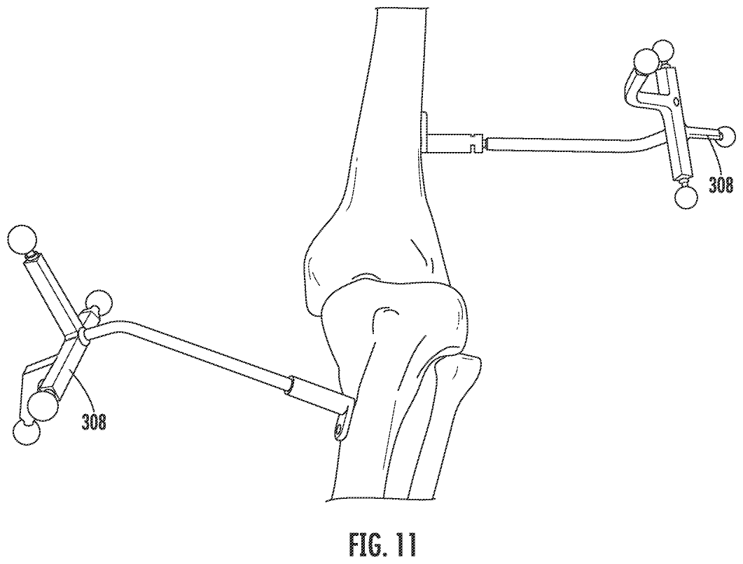

The electronics module 242 as shown in FIGS. 2-5 and 11 processes force data from the force sensors in of the force-measuring device 200. In some examples, the electronics module 242 includes two I2C multiplexers, a Raspberry Pi, and an encoder. The two I2C multiplexers are operably connected to the Raspberry Pi, which receives data from the load cells 236 the encoder in a text file. In some examples such as the example shown in FIG. 11, the electronics module 242 is electrically coupled to the force sensors and includes wiring that extends from each of the force sensors and is attached to a breadboard. In some examples, the electronics module 242 is in operable communication with and receives force data from with the first force sensor 230 and the second force sensor 232 via wires disposed along the dorsal side of the mitt frame 202. In some examples, two I2C multiplexers are connected to a Raspberry Pi. The Raspberry Pi receives data from the load cells 236 and an encoder in a text file. But in other examples, the electronics module 242 can be any suitable configuration to receive data from the force sensors 230, 232 and provide the data to a user. For example, the electronics module 242 can be any combination of electronics that includes one or more breadboards, one or more multiplexers, and one or more computing devices suitable to process force data. In some examples, the electronics module 242 is removably securable to the forearm of a user. Similar to the strap 240 of the force-measuring device 200, In some examples, the force-measuring device 200 includes a strap 244 coupled to the electronics module 242 that removably secure the electronics module 242 to a forearm of the user. The example shown in FIGS. 2-5 includes a hook and loop fastener. But in other examples, the strap can include a clip, a snap, or any other fastener suitable to secure an electronics module 242 about a user's arm, wrist, or hand. Although the electronics module 242 is coupled to other components of the force-measuring device 200, and electrically coupled to the sensors, in some examples the electronics module is wirelessly coupled to the force sensors and can be coupled to other components of the force-measuring device 200 or disposed remote of the mitt frame 202.

In some examples, the electronics module 242 can be used to calculate forces measured by the force sensors 230, 232 in the force-measuring device 200 as described above. For example, the electronics module 242 can sum forces measured by each the four load cells 236 to find the total force applied to the plate (FIG. 28). The equation below can be used by the electronics module to find the total magnitude of force applied to the force plate:

Fₜₒₜₐₗ=FF1+FF2+FF3+FF4

The equations below can be further used to calculate the center of pressure.

$${p}_{x} = [ \frac{- 1}{{F}_{y}} ] [ b ( {F}_{y 1} + {F}_{y 2} - {F}_{y 3} - {F}_{y 4} ) ] \\ {p}_{z} = [ \frac{- 1}{{F}_{y}} ] [ a ( {F}_{y 1} - {F}_{y 2} - {F}_{y 3} + {F}_{y 4} ) ]$$

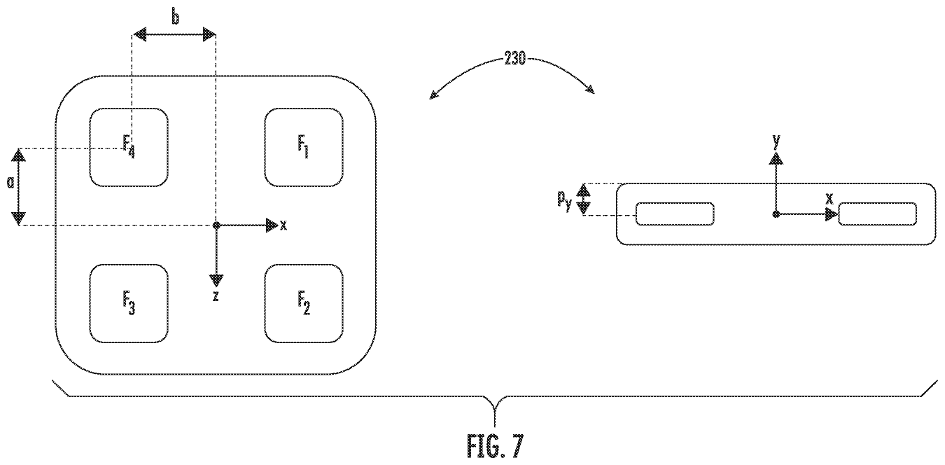

FIG. 7 shows a diagram of force place. F1, F2, F3, and F4 that represent to the four load cells 236 in the force plate. The distance from the origin of the force plate to the center of the force sensor along the x and z axes is represented by a and b (a=b). The distance from the center of the force sensor to the top of the force plate along the y-axis is p_{y} which are used in the electronics module calculations.

In some examples, the force-measuring device 200 also includes a plurality of mitt tracking markers 246 such as the marker shown in FIG. 9. In some examples, each mitt tracking marker 246 is coupled to the mitt frame 202 and disposed on an exterior surface of the mitt frame 202. The electronics module 246 and positional tracking devices such as cameras and promote positional measurement to determine stiffness of a joint being examined. In some examples, the mitt tracking markers 246 are passive optical tracking markers, although in other examples the mitt tracking markers 246 are other markers such as paint, RFID markers or any other device capable of designating location of a portion of a glove. In some examples, the mitt tracking markers 246 are coupled to the finger portion 206 and the palm portion 204. For example, a passive optical tracker can be attached to the dorsal side of the finger portion 206 or the dorsal side of the palm portion 204, depending on how an operator positions their hands and the line of sight of a camera 302 (described in detail below). For example, if a surgeon is applying a force the posterior side of a patient's lower leg with their palm, the dorsal side of the finger part will be better seen by the camera 302 than the palm part that is behind the leg. In contrast, the surgeon could be applying a force to the anterior side of the patient's leg with their palm, and then the dorsal side of the palm will have a better line of sight to the camera 302. In some examples, each mitt tracking marker 246 is positioned such that at least three markers in each mitt tracking marker are visible to a camera 302.

FIG. 1 shows an example system 100 that can be used to determine stability of a joint of a living subject. The system 100 includes the force-measuring device 200 as described above, a displacement-tracking system 300, and a computing device 306. In some examples, he displacement-tracking system 300 is a surgical navigation system that is provided to track position and/or orientation of the force-measuring device and position and/or orientation of one or more body parts associated with the joint of the subject. In some examples, the system 100 is a portable sterilizable system that can be used in an operating room. For example, the system 100 can be used during a surgery or a diagnostic procedure to determine the stability of a joint of a subject such as a knee of a subject. In some examples, the system 100 can be used to determine stiffness and laxity of a knee during an ACLR, TKA, MPLR, MCLR, or LCLR. In some examples, the displacement-tracking system 300 tracks position and orientation of the force-measuring device by tracking position and orientation of the mitt tracking markers 246. The displacement-tracking system 300 may also tracks position and orientation of the one or more body parts associated with the joint of the subject by tracking position and orientation of subject tracking markers 308. In the example shown in FIG. 1, the displacement-tracking system 300 includes a camera 302, a signal processing unit 304, and a computing device 306 operably coupled to the camera 302 and the signal processing unit 304, a first and second subject tracking marker 308 to track the position and orientation of body parts relative to each other. In the example shown in FIG. 1, the camera is an NDI Polaris Spectra. The camera 302 may track each passive optical tracker and communicate with the computing device 306 to record the tracker's position and orientation. The camera 302 may track passive optical tracking markers with a linear accuracy of at least 2 mm and worst-case angular accuracy of 1.25 degrees. The sampling rate of the navigation system can be set at 20, 40, or 60 Hz. In other examples, the camera can be any camera suitable to track moving objects in an operating room and relay positional data to a processor.

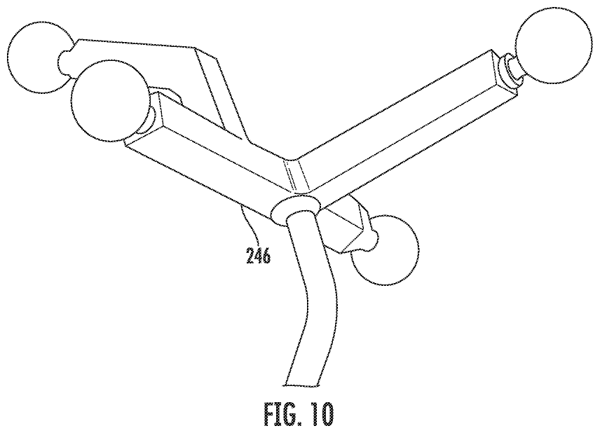

In some examples, the first subject tracking marker 308 and the second subject tracking marker 308 are passive optical tracking markers. Although in other examples, the subject tracking markers are other markers such as paint, MD markers or any other device capable of designating location of a body part in relation to another body part. In some examples, the first subject tracking marker 308 can be removably coupled to a first body part associated with a joint of a subject that is to be assessed and the second subject tracking marker 308 can be removably coupled to a second body part associated with the joint of the subject. For example, each subject tracking marker 308 can be fastened to bones of a patient such as a tibia or femur using fasteners such as bone pins or screws as shown in FIG. 10.

In some examples, the computing device 306 is a processor in operable communication with the force-measuring device and the displacement-tracking system. As described above, in some examples, the computing device is operably coupled to the force-measuring device 200 and the displacement-tracking system 300 to receive force data from the force-measuring device 200 and position and orientation data from the displacement-tracking system. The subject position and orientation data are indicative of positions and orientations of the one or more body parts associated with the joint of the subject. In some examples, the computing device 306 determines subject displacement data indicative of rotational and translational displacements of the one or more body parts associated with the joint of the subject by using the subject position and orientation data.

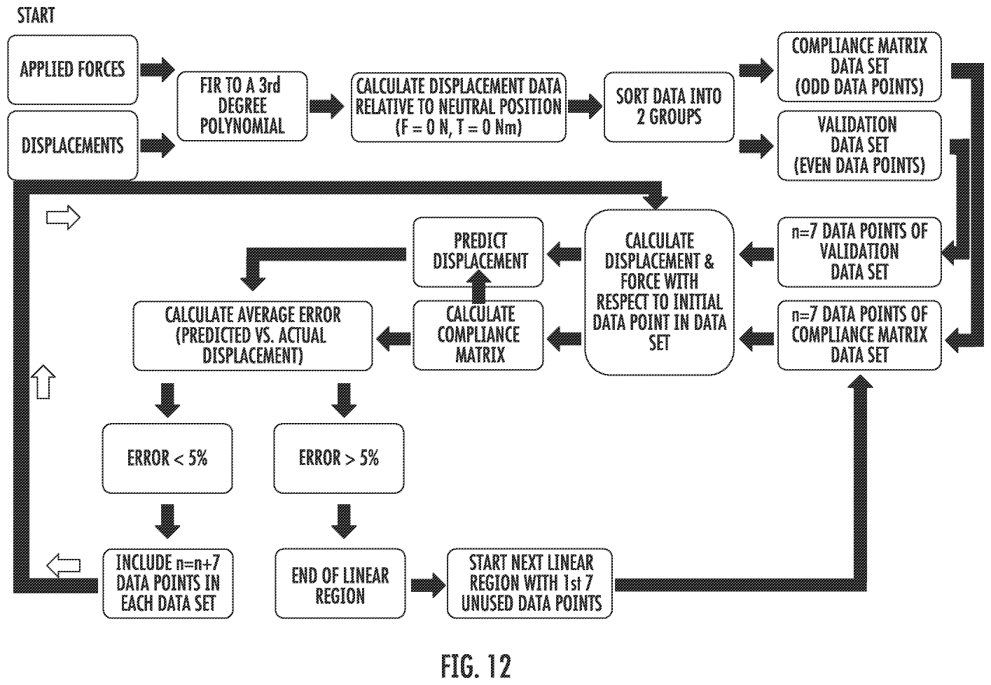

In some examples, the computing device 306 is further operably coupled to the force-measuring device 200 to determine various values. In some examples, the computing device 306 receives force data from the force-measuring device 200, where force data is indicative of forces applied by the user to the joint of the subject via the force-measuring device. In some examples, the computing device 306 is further operably coupled to the displacement-tracking system 300 to receive mitt position and orientation data from the displacement-tracking system 300. In some examples, the mitt position and orientation data are indicative of positions and orientations of the mitt frame 202. In some examples, the computing device 306 can use the force data and the mitt position and orientation data, to determine moment data indicative of moments about the joint of the subject resulting from the forces applied by the user. Further, in some examples, the computing device 306 receives position and orientation information from the camera 302 about each passive optical tracker relative to the camera 302. In some examples, the position and orientation data for each passive optical tracker 308 are transformed into terms of tibia and femur coordinate systems defined by anatomical landmarks. The rotations and translations of the knee can be found based on an established knee coordinate system. In some examples, this data is used in conjunction with the force mitt data to determine knee stability using a custom knee stability algorithm such as the example algorithm shown in the diagram of FIG. 12. In some examples, the computing device determines one or more stability values indicative of stability of the joint of the subject, based at least in part on the subject displacement data, the force data, and the moment data.

Although in some examples, the displacement-tracking system 300 includes a camera 302, a signal processing unit 304, and a computing device 306, n some examples, the system 300 includes any number of cameras signal processing units 304, or computing devices, suitable to track and process movement of the gloves or body parts. Although in some examples, the displacement-tracking system 300 is a surgical navigation system, in some examples the displacement-tracking system 300 is any other system suitable for tracking the position of objects in an operating room. In some examples, the displacement-tracking system 300 uses an RFD sensor, an infrared sensor, or any other sensor suitable to determine the position of objects in an operating room.

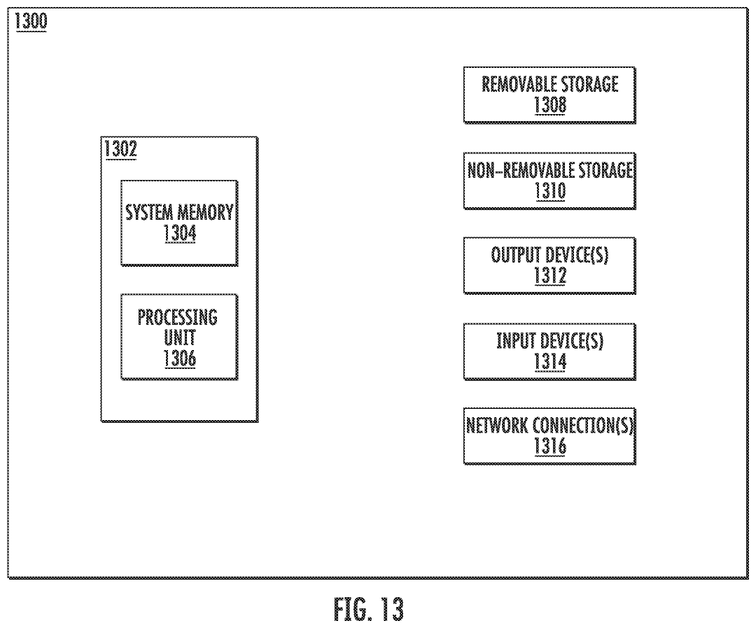

It should be appreciated that the logical operations described herein with respect to the various figures may be implemented (1) as a sequence of computing device implemented acts or program modules (i.e., software) running on a computing device (e.g., the computing device described in FIG. 13), (2) as interconnected machine logic circuits or circuit modules (i.e., hardware) within the computing device and/or (3) a combination of software and hardware of the computing device. Thus, the logical operations discussed herein are not limited to any specific combination of hardware and software. The example is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 13, an example computing device 1300 upon which the methods described herein may be implemented is illustrated. It should be understood that the example computing device 1300 is only one example of a suitable computing environment upon which the methods described herein may be implemented. As described above, this disclosure contemplates that the computing device 306 can include a microprocessor. Such a microprocessor can be made durable and/or protected to handle the high shock and vibration generated upon impact with a hardened target. Optionally, the computing device 1300 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media. As described above, this disclosure contemplates that the computing device 306 can include a microprocessor.

In its most basic configuration, computing device 1300 typically includes at least one processing unit 1306 and system memory 1304. Depending on the exact configuration and type of computing device, system memory 1304 may be volatile (such as random access memory (RAM)), non-volatile (such as read-only memory (ROM), flash memory, etc.), or some combination of the two. This most basic configuration is illustrated in FIG. 13 by dashed line 1302. The processing unit 1306 may be a standard programmable processor that performs arithmetic and logic operations necessary for operation of the computing device 1300. The computing device 1300 may also include a bus or other communication mechanism for communicating information among various components of the computing device 1300.

Computing device 1300 may have additional features/functionality. For example, computing device 1300 may include additional storage such as removable storage 1308 and non-removable storage 1310 including, but not limited to, magnetic or optical disks or tapes. Computing device 1300 may also contain network connection(s) 1316 that allow the device to communicate with other devices. Computing device 1300 may also have input device(s) 1314 such as a keyboard, mouse, touch screen, etc. Output device(s) 1312 such as a display, speakers, printer, etc. may also be included. The additional devices may be connected to the bus in order to facilitate communication of data among the components of the computing device 1300. All these devices are well known in the art and need not be discussed at length here.

The processing unit 1306 may be configured to execute program code encoded in tangible, computer-readable media. Tangible, computer-readable media refers to any media that is capable of providing data that causes the computing device 1300 (i.e., a machine) to operate in a particular fashion. Various computer-readable media may be utilized to provide instructions to the processing unit 1306 for execution. Example tangible, computer-readable media may include, but is not limited to, volatile media, non-volatile media, removable media and non-removable media implemented in any method or technology for storage of information such as computer readable instructions, data structures, program modules or other data. System memory 1304, removable storage 1308, and non-removable storage 1310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example, the processing unit 1306 may execute program code stored in the system memory 1304. For example, the bus may carry data to the system memory 1304, from which the processing unit 1306 receives and executes instructions. The data received by the system memory 1304 may optionally be stored on the removable storage 1308 or the non-removable storage 1310 before or after execution by the processing unit 1306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware examples.

Although examples have been described in language specific to structural features and/or methodological acts, it is to be understood that the disclosure is not necessarily limited to the specific features or acts described. Rather, the specific features and acts are disclosed as illustrative forms of implementing the described subject matter. Conditional language, such as, among others, “can,” “could,” “might,” or “may,” unless specifically stated otherwise, or otherwise understood within the context as used, is generally intended to convey that certain examples could include, while other examples do not include, certain features, elements, and/or steps. Thus, such conditional language is not generally intended to imply that features, elements, and/or steps are in any way required for one or more examples.

1. A force-measuring device for determining stability of a joint of a living subject, the force-measuring device comprising:
a mitt frame configured to receive a hand of a user therein, the mitt frame comprising:
a palm portion configured to receive a palm of the user therein; and
a finger portion configured to receive fingers of the user therein;
one or more first force sensors coupled to the palm portion and disposed on an exterior surface of the palm portion; and
one or more second force sensors coupled to the finger portion and disposed on an exterior surface of the finger portion.

2. (canceled)

3. The force-measuring device of claim 1, wherein the finger portion is movably coupled to the palm portion.

4. The force-measuring device of claim 3, wherein the finger portion is rotatably coupled to the palm portion.

5. The force-measuring device of claim 4, further comprising a rotary encoder coupled to the finger portion and the palm portion and configured to track a location of the finger portion relative to the palm portion.

6. (canceled)

7. The force-measuring device of claim 1, wherein the palm portion comprises a palmar component and a dorsal component coupled to one another.

8. The force-measuring device of claim 7, wherein the palmar component of the palm portion is formed of a first material, wherein the dorsal component of the palm portion is formed of a second material, and wherein the first material is more flexible than the second material.

9. The force-measuring device of claim 7, wherein the palmar component of the palm portion comprises one or more pockets, and wherein the one or more first force sensors are disposed at least partially within the one or more pockets.

10. (canceled)

11. (canceled)

12. The force-measuring device of claim 1, wherein a palmar component of the finger portion is formed of a first material, wherein a dorsal component of the finger portion is formed of a second material, and wherein the first material is more flexible than the second material.

13. The force-measuring device of claim 1, wherein a palmar component of the finger portion comprises one or more pockets, and wherein the one or more second force sensors are disposed at least partially within the one or more pockets.

14. (canceled)

15. The force-measuring device of claim 1, wherein the one or more first force sensors are disposed on a palmar side of the palm portion.

16. (canceled)

17. The force-measuring device of claim 1, wherein the one or more first force sensors or the one or more second force sensors comprise one or more load cells.

18. The force-measuring device of claim 1, wherein the one or more first force sensors or the one or more second force sensors comprise one or more piezoelectric sensors.

19. The force-measuring device of claim 1, wherein the one or more first force sensors or the one or more second force sensors comprise one or more piezoresistive sensors.

20. The force-measuring device of claim 1, wherein the one or more first force sensors or the one or more second force sensors comprise one or more force plates.

21. The force-measuring device of claim 20, wherein the one or more force plates comprise one or more load cells disposed between a pair of plates.

22. The force-measuring device of claim 1, wherein the one or more second force sensors are disposed on a palmar side of the finger portion.

23-33. (canceled)

34. The force-measuring device of claim 1, further comprising a plurality of mitt tracking markers coupled to the mitt frame and disposed on an exterior surface of the mitt frame.

35. (canceled)

36. (canceled)

37. The force-measuring device of claim 34, wherein the mitt tracking markers comprise passive optical tracking markers.

38-40. (canceled)

41. The force-measuring device of claim 1, further comprising a rotary encoder coupled to a thumb portion and the palm portion and configured to track a location of the thumb portion relative to the palm portion.

42. (canceled)

43. The force-measuring device of claim 1, further comprising one or more third force sensors coupled to a thumb portion and disposed on an exterior surface of the thumb portion.

44-82. (canceled)
